(19) United States
(12) Patent Application Publication
(10) Pub. No.: US 2011/0071383 A1
(43) Pub. Date: Mar. 24, 2011

(54) VISUALIZATION OF ABNORMALITIES OF AIRWAY WALLS AND LUMENS

(75) Inventors: Carol L. Novak, Newtown, PA (US); Benjamin L. Odry, West New York, NJ (US)

(73) Assignee: Siemens Corporation, Iselin, NJ (US)

(21) Appl. No.: 12/794,825

(22) Filed: Jun. 7, 2010

Related U.S. Application Data
(60) Provisional application No. 61/244,986, filed on Sep. 23, 2009.

(51) Int. Cl. A61B 5/055; G06T 15/00; G06G 7/48

(52) U.S. Cl. 600/416; 345/419; 703/11

(57) ABSTRACT

An exemplary embodiment of the present invention includes a method of visualizing an airway of a bronchial tree. The method includes generating a tree model from an airway segmentation of a bronchial tree, determining a lumen, a wall thickness, and an adjacent artery for a branch of the tree model, determining whether the lumen of the branch has a first abnormal state and the wall thickness of the branch has a second abnormal state based on the adjacent artery, and illustrating the branch in one of a plurality of visually distinct styles based on the first and second abnormal states.

### CROSS-REFERENCE TO RELATED APPLICATIONS

This application claims priority to U.S. Provisional Application No. 61/244,986, filed on Sep. 23, 2009, the disclosure of which is incorporated by reference herein.

### BACKGROUND OF THE INVENTION

1. Technical Field

The present disclosure relates to medical image processing and display, and more particularly, to evaluation and display of abnormalities of airways.

2. Discussion of Related Art

Pulmonary diseases such as bronchiectasis, asthma, cystic fibrosis and Chronic Obstructive Pulmonary Disease (COPD) may be characterized by abnormalities in airway dimensions, including the thickness of the walls and the size of the inner airway space (e.g., referred to as a lumen). Computed Tomography (CT) has become one of the primary means to depict and detect these abnormalities, as the availability of high-resolution near-isotropic data makes it possible to evaluate airways at oblique angles to the scanner plane. However, currently, clinical evaluation of the airways is typically limited to subjective visual inspection.

When the airways are affected by disease, the inner airway space (lumen) can be narrowed or dilated, and/or the airway wall (e.g., the outer ring) can be thickened. One can determine whether an airway is abnormal by computing a ratio of the area or diameter of the lumen of the airway to the area or diameter of the wall of the airway. However, an airway having both an abnormal lumen and an abnormal bronchial wall thickness may be give rise to ratio that is identical to an airway with a normal lumen and a normal bronchial wall thickness.

Thus, there is a need for method and systems that can more accurately evaluate and visualize abnormalities of airways.

### SUMMARY OF THE INVENTION

According to an exemplary embodiment of the present invention, a method of visualizing an airway of a bronchial tree includes generating a tree model from an airway segmentation of the bronchial tree, determining a lumen, a wall thickness, and an adjacent artery for a branch of the tree model, determining whether the lumen of the branch has a first abnormal state and the wall thickness of the branch has a second abnormal state based on the adjacent artery, and illustrating the branch in one of a plurality of visually distinct styles based on the first and second abnormal states.

The illustrating may include drawing the branch in a first style when only the first abnormal state is present, drawing the branch in a second style when only the second abnormal state is present, drawing the branch in both the first style and the second style when both the first and second abnormal states are present, and drawing the branch in a third style when neither of the states is present.

The first style may be a first color, the second style may be a first line thickness, the third style may be a second color and a second line thickness, where the second color is different from the first color and the second line thickness is thinner than the first line thickness. Alternately, the second style may be a first color, the first style may be a first line thickness, the third style may be a second color and a second line thickness, where the second color is different from the first color and the second line thickness is thinner than the first line thickness.

According to an exemplary embodiment of the present invention, a method of visualizing an airway of a bronchial tree includes generating a tree model from an airway segmentation of a bronchial tree, determining whether a lumen of a branch of the tree model has a first abnormal state by one of comparing a size of the lumen to an absolute size or comparing a size of the lumen to a size scaled according to a generation of the branch, determining whether a wall thickness of the branch has a second abnormal state by one of comparing the wall thickness to an absolute thickness or comparing the wall thickness to a thickness scaled according to a generation of the branch, and illustrating the branch in one of a plurality of visually distinct styles based on the first and second abnormal states.

According to an exemplary embodiment of the present invention, a system for displaying an airway in a bronchial tree includes a display, a memory device for storing a program, a processor in communication with the memory device, the processor operative with the program to generate a tree model from an airway segmentation of a bronchial tree, determine a lumen, a wall thickness, and an adjacent artery for a branch of the tree model, determine whether the lumen of the branch has a first abnormal state and the wall thickness of the branch has a second abnormal state based on the adjacent artery, and illustrate the branch on the display in one of a plurality of visually distinct styles based on the first and second abnormal states.

The system may further include an acquisition device to acquire a 3D image of the bronchial tree from a patient for generating the airway segmentation. The acquisition device may be a multi-slice computed tomography (MSCT) imaging device or a magnetic resonance (MR) scanner.

### BRIEF DESCRIPTION OF THE DRAWINGS

Exemplary embodiments of the invention can be understood in more detail from the following descriptions taken in conjunction with the accompanying drawings in which:

FIG. 1 is a block diagram illustrating a system for displaying an airway according to an exemplary embodiment of the present invention.

FIG. 2 is a flowchart illustrating a method for airway visualization according to an exemplary embodiment of the present invention.

FIGS. 3A-E illustrates exemplary visualizations that may be produced by the method of FIG. 2 according to an exemplary embodiment of the present invention.

FIG. 4 illustrates an example of a computer system capable of implementing the methods and systems according to embodiments of the present invention.

### DETAILED DESCRIPTION OF EXEMPLARY EMBODIMENTS

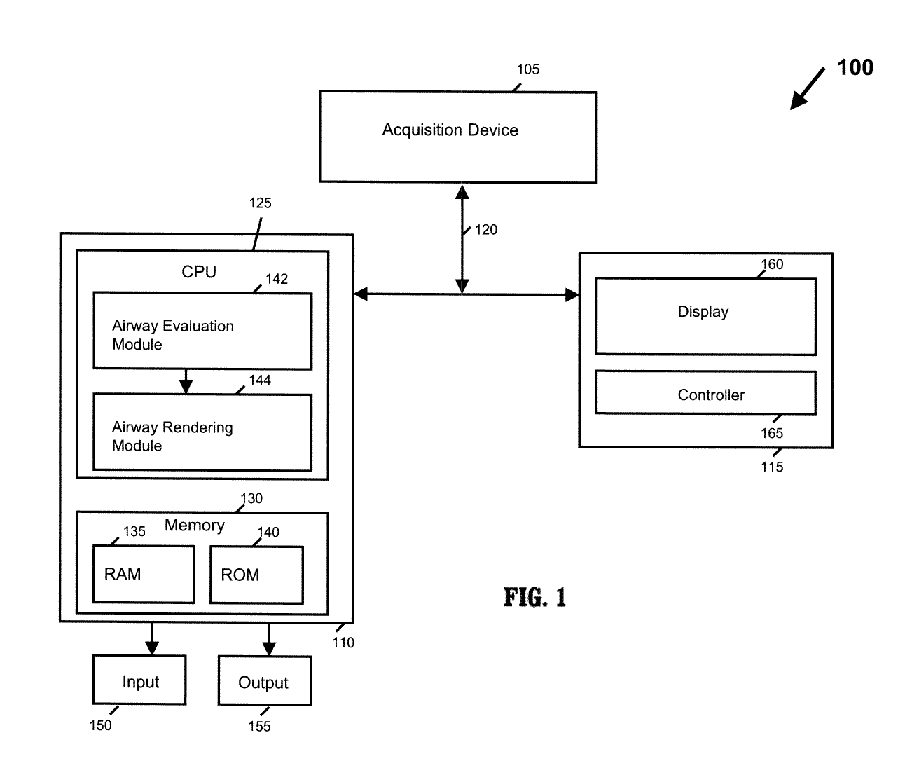

In general, exemplary embodiments of systems and methods for displaying an airway will now be discussed in further detail with reference to FIGS. 1-4. This invention may, however, be embodied in different forms and should not be construed as limited to the embodiments set forth herein. Rather, these embodiments are provided so that this disclosure will be thorough and complete, and will fully convey the scope of the invention to those skilled in the art.

It is to be understood that the systems and methods described herein may be implemented in various forms of hardware, software, firmware, special purpose processors, or a combination thereof. In particular, at least a portion of the present invention may be implemented as an application comprising program instructions that are tangibly embodied on one or more program storage devices (e.g., hard disk, magnetic floppy disk, RAM, ROM, CD ROM, etc.) and executable by any device or machine comprising suitable architecture, such as a general purpose digital computer having a processor, memory, and input/output interfaces. It is to be further understood that, because some of the constituent system components and process steps depicted in the accompanying Figures may be implemented in software, the connections between system modules (or the logic flow of method steps) may differ depending upon the manner in which the present invention is programmed. Given the teachings herein, one of ordinary skill in the related art will be able to contemplate these and similar implementations of the present invention.

FIG. 1 is a block diagram illustrating a system 100 for displaying an airway according to an exemplary embodiment of the present invention. As shown in FIG. 1 the system 100 includes an acquisition device 105, a PC 110 and an operator's console 115 connected over a wired or wireless network 120. In an alternate embodiment, the devices are directly connected to one another without use of network 120.

The acquisition device 105 may be a multi-slice computed tomography (MSCT) imaging device or any other three-dimensional (3D) high resolution imaging device such as a magnetic resonance (MR) scanner.

The PC 110, which may be a portable or laptop computer, a medical diagnostic imaging system or a picture archiving communications system (PACS) data management station, includes a CPU 125 and a memory 130 connected to an input device 150 and an output device 155. The CPU 125 includes an airway evaluation module 142 that includes one or more methods to evaluate whether an airway is normal or has one or more abnormalities. The CPU 125 further includes an airway rendering module 144 that includes one or more methods for rendering an airway for visualization of whether the airway is normal or includes the one or more abnormalities on display 160. Although shown inside the CPU 125, the airway evaluation module 142 and/or the airway rendering module 144 can be located outside the CPU 125. Further, although the airway evaluation module 142 and airway rendering modules 144 are shown as being separate modules, they may be included within the same module.

The memory 130 includes a RAM 135 and a ROM 140. The memory 130 can also include a database, disk drive, tape drive, etc., or a combination thereof. The RAM 135 functions as a data memory that stores data used during execution of a program in the CPU 125 and is used as a work area. The ROM 140 functions as a program memory for storing a program executed in the CPU 125. The input 150 is constituted by a keyboard, mouse, etc., and the output 155 is constituted by an LCD, CRT display, printer, etc.

The operation of the system 100 can be controlled from the operator's console 115, which includes a controller 165, e.g., a keyboard, and the display 160. The operator's console 115 communicates with the PC 110 and the acquisition device 105 so that image data collected by the acquisition device 105 can be rendered by the PC 110 and viewed on the display 160. It is to be understood that the PC 110 can be configured to operate and display information provided by the acquisition device 105 absent the operator's console 115, using, e.g., the input 150 and output 155 devices to execute certain tasks performed by the controller 165 and the display 160.

The operator's console 115 may further include any suitable image rendering system/tool/application that can process digital image data of an acquired image dataset (or portion thereof) to generate and display images on the display 160. More specifically, the image rendering system may be an application that provides rendering and visualization of medical image data, and which executes on a general purpose or specific computer workstation. It is to be understood that the PC 110 can also include the above-mentioned image rendering system/tool/application.

A 3D image data of a bronchial tree may acquired from a patient by using the acquisition device 105, which is operated at the operator's console 115, to scan the patient's chest thereby generating a series of two-dimensional (2D) image slices associated with the chest. The 2D image slices are then combined to form a 3D image (e.g., a volume) of the bronchial tree, which can be displayed on the display 160 and/or sent to the airway evaluation module 142 for evaluation of abnormalities. However, the system 100 need not include the acquisition device 105. For example, the image data of the bronchial tree could be predefined on the PC 110, sent to the PC 110 across the network 120, or loaded from a removable storage medium such as a CD, USB drive, etc.

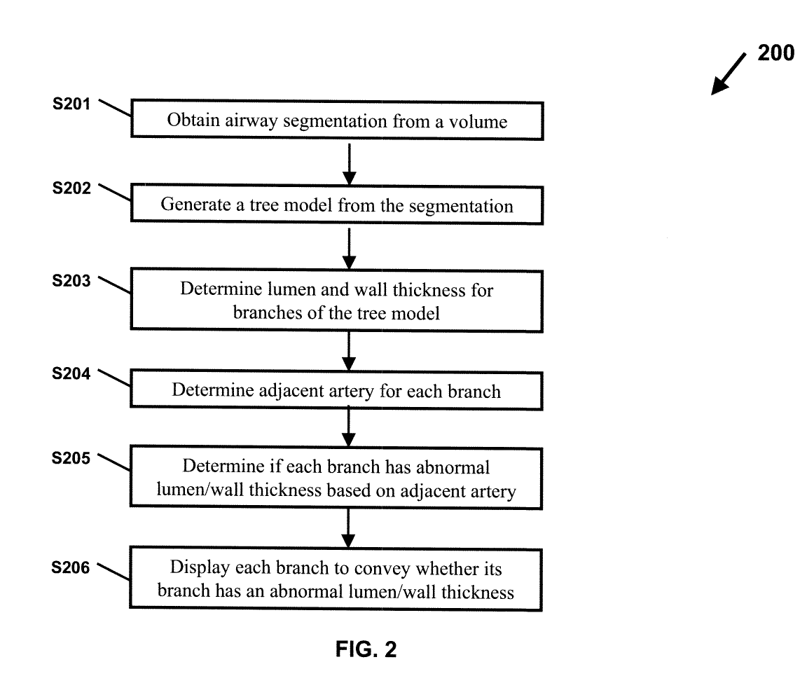

FIG. 2 is a flowchart showing an operation of a method for airway visualization according to an exemplary embodiment of the present invention. Referring to FIG. 2, an airway segmentation image Iₛ is obtained from a volume I (e.g., from a CT scan) (S201), a tree model T is generated from the segmentation image Iₛ (S202), an airway lumen and airway wall thickness is determined for each branch of the tree model (S203), an adjacent artery corresponding to each branch is determined (S204), a determination is made whether each branch has an abnormal lumen and/or wall thickness based on the ratio to the corresponding adjacent artery (S205), and each branch is displayed to convey whether its lumen and its wall thickness is normal or abnormal (S206). The airway evaluation module 142 may be used to performed steps (S201-205) and the airway rendering module 144 may be use to perform step (S206). The step (S204) of determining the arteries may be performed before the lumens and wall thicknesses are determined. Embodiments of steps (S201-S206) will be described in more detail below.

The step S201 of obtaining an airway segmentation image Iₛ from a volume I can be performed using any number of suitable segmentation techniques. For example, a bronchial tree of the volume I can be segmented by using the technique described in Kiraly A. P., McLennan G., Hoffman E. A., Reinhardt J. M., and Higgins W. E., (2002) “Three-dimensional human airway segmentation methods for clinical virtual bronchoscopy” Academic Radiology, 2002. 9(10): p. 1153-1168. The step S202 of generating a tree model T from the segmentation image Iₛ may be performed using a method described in Kiraly A. P., Helferty J. P., Hoffman E. A., McLennan G. and Higgins W. E. (2004) “Three dimensional path planning for virtual bronchoscopy” in IEEE Transactions on Medical Imaging, vol. 23, no. 1, November 2004: p. 1365-1379. The method is based on a skeletonization followed by refinement steps to create a smooth tree model of the segmented airway tree. The result is a hierarchical description of the tree as a connected series of branches. Each branch is described by a series of sites. In addition to containing positional information, each site also contains orientation information of the branch at that point.

The step S203 of determining an airway lumen and thickness (e.g., a wall extent) for each branch of the airway tree may be performed by various methods for computing the lumen and wall extent. Examples of computing a lumen and a wall extent are discussed in Odry B., Kiraly A. P., Slabaugh G. and Novak C. L. (2008) “Active contour approach for accurate quantitative airway analysis” in SPIE Medical Imaging, vol. 6916, 2008: p. 691-613 or Kiraly A. P., Odry B. L., Naidich P. and Novak C. L. (2007) “Boundary-Specific Cost Functions for Quantitative Airway Analysis” in Medical Image Computing and Computer Assisted Intervention (MICCAI) 2007: p. 784-791.

The skeletonization method determines for each airway branch the centerline and its orientation. Thus, this information can be used to determine cross-sectional views at any point within the airway branch. Once the voxels that form the lumen have been identified on a given cross section, measurements of the lumen can be derived. For examples, the lumen measurements may include average lumen diameter, minimum lumen diameter, maximum lumen diameter, and lumen area. Similarly, once the voxels that form the wall have been identified, measurements of the wall can be derived. For example, the wall measurements may include average wall thickness, minimum wall thickness, maximum wall thickness, and wall area.

The step (S204) of determining an adjacent artery corresponding to each branch refers to a step of determining the corresponding artery that accompanies each airway in the lungs. The diameters of healthy airways may vary based on generation number, with the airways decreasing as the airway generation increases. Similarly, the diameter of arteries may decrease as the generation increases. In healthy lungs, the diameter of an airway should be roughly equivalent to the diameter of its accompanying artery. If the airway diameter is significantly larger than the artery, this may indicate that the patient's airways are abnormally dilated.

The step (S205) of determining whether each airway lumen and wall thickness is abnormal based on the corresponding adjacent artery may involve calculation of a Bronchial Lumen to Artery (BLA) ratio and a Bronchial Wall to Artery (BWA) ratio.

A normal airway should have an airway lumen of about the same size (e.g., diameter) as its corresponding artery, giving rise to a BLA ratio of about 1.0. The BLA ratio may vary slightly from 1.0 and still be classified as normal (e.g., + or −1%, 5%, 10%, etc.). A normal airway should also have a wall thickness about one quarter the diameter of the adjacent artery, giving rise to a BWA ratio of about 0.25 or less. The upper limit of the BWA ratio for a normal wall thickness may vary slightly from 0.25 and still be classified as normal (e.g., + or −1%, 5%, 10%, etc.)

The step (S206) of displaying each airway branch to convey whether its lumen and its wall thickness is abnormal may involve assigning a color code and a line thickness code to each branch based on whether the lumen and/or the wall thickness of the branch is abnormal or normal, and rendering the airways from the determined branches and their assigned color and line thickness codes.

In at least one embodiment of the present invention, if a lumen of a branch is normal the corresponding branch is displayed in a first color (e.g., green), or if the lumen of a branch is abnormal the corresponding branch is displayed in a second and different color (e.g., red, blue, orange, yellow, etc.). In at least one embodiment of the present invention, if a wall thickness of a branch is normal, the branch can be displayed with a normal line style, or if the thickness of a wall is abnormal, the branch can be displayed with a thicker line style.

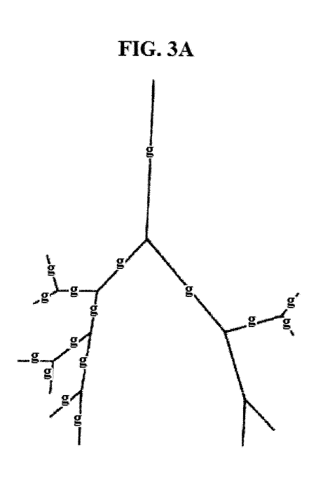

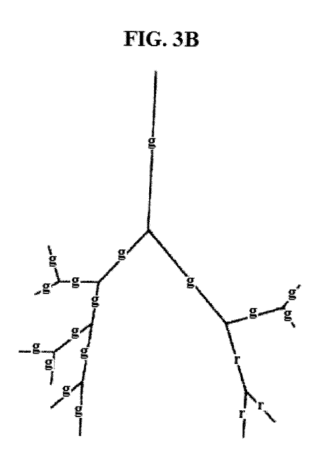

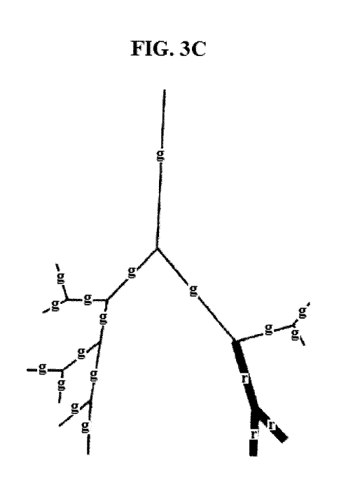

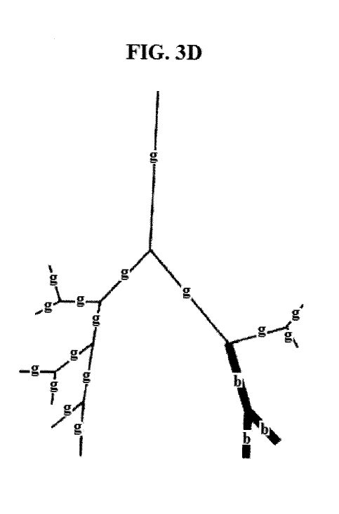

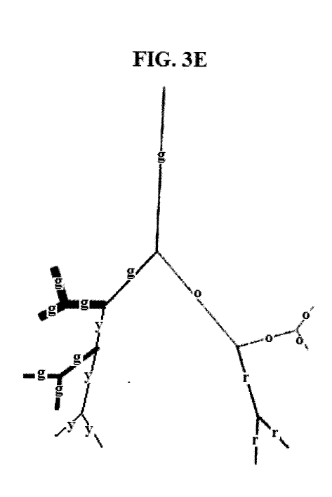

FIG. 3A depicts an example of an idealized normal bronchial tree. All branches are colored in green and are shown as thin lines to indicate that all bronchi have both normal lumen diameters and normal wall thicknesses.

FIG. 3B depicts an example of a bronchial tree where the branches in the lower left lobe (LLL, on the right side of the image) are colored red. However, all the branches still have a thin line width. This indicates that while the airway lumen is abnormally dilated in the LLL, the walls have a normal thickness.

FIG. 3C depicts an example of a bronchial tree where the LLL is affected both by airway lumen dilation, as indicated by the red coloring, and by airway wall thickening, as indicated by the thickened lines for those branches.

FIG. 3D depicts an example of a bronchial tree where the LLL is affected by airway lumen narrowing, as indicated by the blue coloring, and by airway wall thickening, as indicated by the thickened lines for those branches.

Besides an illustration of a binary determination of normal or abnormal, it is possible to depict a tree with quantized color and thicknesses. For example, additional colors or finer gradations of the colors may be used to further distinguish between branches whose lumens are mildly enlarged, moderately enlarged, severely enlarged, mildly narrowed, moderately narrow, or severely narrowed. A mildly enlarged lumen may have a BLA ranging between 1.0 and 2.0, a moderately enlarged lumen may have a BLA ranging between 2.0 and 3.0, a severely enlarged lumen may have a BLA ranging greater than about 3.0, and a narrowed lumen may have a BLA ratio below about 0.8. These BLA ratio values may vary slightly and still be classified as respectively mildly enlarged, moderately enlarged, severely enlarged, or narrowed (e.g., + or −1%, 5%, 10%, etc.).

Additional line thicknesses may be used to distinguish between branches whose wall thicknesses are mildly thickened, moderately thickened, severely thickened, mildly thinned, moderately thinned, or severely thinned. For example, a mildly thickened airway wall may have a BWA ranging between 0.25 and 0.5, a moderately thickened airway wall may have a BWA ranging between 0.5 and 1.0, and a severely thickened airway wall may have a BWA greater than about 1.0. These BWA ratio values may vary slightly and still be classified as being respectively mildly, moderately, or severely (e.g., + or −1%, 5%, 10%, etc.).

For example, FIG. 3E depicts an example of a bronchial tree where the right upper lobe (RUL) includes severely thickened airways walls and normal lumens (see e.g., thickest lines colored green), the right middle lobe (RML) includes moderately thickened airway walls and normal lumens (see e.g., thick lines colored green), the right lower lobe (RLL) includes normal airway walls and mildly dilated lumens (see e.g., normal thickness lines colored yellow), the left upper lobe (LUL) includes normal airway walls and moderately dilated lumens (see e.g., normal thickness lines colored orange, and the LLL includes normal airway walls and severely dilated lumens (see e.g., normal thickness lines colored red), and the trachea and left and right main bronchi have normal lumens and wall thicknesses (e.g., see normal thickness lines colored green).

In an alternate embodiment, the method can be varied to have the coloring code different levels of wall thickening and the line thickness code different levels of lumen diameter. Further, parameters may be set or selections may be made to focus only on line coloring or on line thickness. For example, if a user is only interested in wall thickness abnormalities, lumen abnormalities may be hidden, or vice versa. Further, parameters may be set or selections may be made to indicate whether lumen abnormalities will be visualized using color coding and the thickness abnormalities will be visualized using line thickness coding or vice versa. In addition, parameters may be set or selections may be made to focus only on a certain portion of the bronchial tree. For example, a user may set a parameter or a selection to indicate RUL, RML, RLL, LUL, LLL, etc.

Determining whether an airway lumen and wall thickness of a branch is abnormal need not be determined based on the corresponding adjacent artery. For example, the size of a lumen of a branch can be compared to one or more predefined absolute sizes to determine whether the lumen is normal, mildly enlarged, moderately enlarged, severely enlarged, or narrowed. Similarly, the wall thickness of a branch can be compared to one or more predefined absolute thicknesses to determine whether the thickness of the branch is normal, mildly thickened, moderately thickened, or severely thickened. Further, the size of a lumen of a branch can be compared to one or more predefined sizes scaled according to the generation of the branch to determine whether the lumen is normal, mildly enlarged, moderately enlarged, severely enlarged, or narrowed. Similarly, the wall thickness of a branch can be compared to one or more predefined thicknesses scaled according to the generation of the branch to determine whether the thickness of the branch is normal, mildly thickened, moderately thickened, or severely thickened.

Not all branches of a bronchial tree need be examined for abnormalities. For example, a parameter may be set or a selection may be made to specify that abnormalities only be evaluated in certain regions (e.g., LLL, RML, etc.). Further, a parameter may be set or a selection may be made to specify that certain regions be evaluated only for lumen abnormalities or only for wall thickness abnormalities.

Some branches of a bronchial tree may be determined to be abnormal (e.g., in lumen and/or wall thickness) using the above described BLA and/or BWA ratio values, while other branches of the same bronchial tree may use the above described predefined absolute sizes and thicknesses or the sizes and thicknesses scaled based on the generation of the branch.

Please note that each branch in FIGS. 3A-E is labeled with a character (e.g., g for green, r for red, y for yellow, o for orange, b for blue, etc.) merely to indicate the color that branch would be visualized on a color display (e.g., 160) because the figures are presented in black and white in the application.

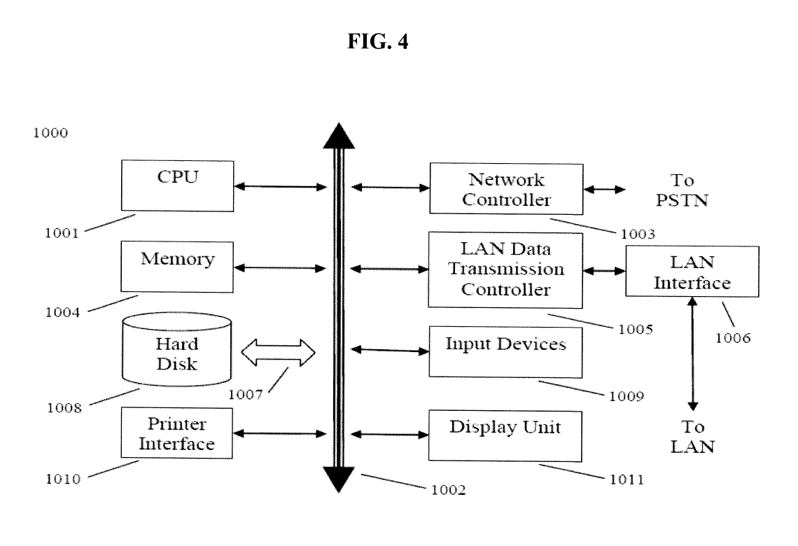

FIG. 4 shows an example of a computer system, which may implement a method and system of the present disclosure. The system and methods of the present disclosure, or part of the system and methods, may be implemented in the form of a software application running on a computer system, for example, a mainframe, personal computer (PC), handheld computer, server, etc. For example, the airway evaluation module 142 and the airway reconstruction module 144 of FIG. 1 and the method of FIG. 2 may be implemented as software application(s). Further, the method of FIG. 2 may also be performed by applications. These software applications may be stored on a computer readable media (such as hard disk drive memory 1008) locally accessible by the computer system and accessible via a hard wired or wireless connection to a network, for example, a local area network, or the Internet.

The computer system referred to generally as system 1000 may include, for example, a central processing unit (CPU) 1001, a GPU (not shown), a random access memory (RAM) 1004, a printer interface 1010, a display unit 1011, a local area network (LAN) data transmission controller 1005, a LAN interface 1006, a network controller 1003, an internal bus 1002, and one or more input devices 1009, for example, a keyboard, mouse etc. As shown, the system 1000 may be connected to a data storage device, for example, a hard disk, 1008 via a link 1007. CPU 1001 may be the computer processor that performs some or all of the steps of the methods described above with reference to FIGS. 1-3.

Embodiments of the present image are not limited to images of any particular format, size, or dimension. For example, the above methods and system may be applied to images of various imaging formats such as magnetic resonance image (MRI), computed tomography (CT), positron emission tomography (PET), etc.

At least one of above described exemplary embodiments can show important diagnostic information (e.g., in a single glance), which may be used by a healthcare provider (e.g., a physician) to diagnose a medical condition. For example, a healthcare provider may determine whether the airways are abnormal, where the airways are normal (e.g. upper lobes vs. lower lobes or right side vs. left side), how much of the lungs are affected, how severe are the abnormalities, are both the lumens and bronchial walls affected, whether there is a correspondence between wall thickening and lumen abnormalities, etc. Further, by depicting both wall thickening and lumen abnormalities in the same image, a healthcare provider can quickly appreciate the degree to which the wall thickening and lumen abnormalities are correlated. For example, examination of wall thickening independent of lumen abnormalities may result in an incorrect diagnosis, which could drive an incorrect treatment decision.

Although the illustrative embodiments have been described herein with reference to the accompanying drawings, it is to be understood that the present invention is not limited to those precise embodiments, and that various other changes and modifications may be affected therein by one of ordinary skill in the related art without departing from the scope or spirit of the invention. All such changes and modifications are intended to be included within the scope of the invention.

What is claimed is:

1. A method of visualizing an airway of a bronchial tree, the method comprising:
generating, by a processor, a tree model from an airway segmentation of the bronchial tree;
determining, by a processor, a lumen, a wall thickness, and an adjacent artery for a branch of the tree model;
determining, by the processor, whether the lumen of the branch has a first abnormal state and the wall thickness of the branch has a second abnormal state based on the adjacent artery; and
illustrating the branch in one of a plurality of visually distinct styles based on the first and second abnormal states.

2. The method of claim 1, wherein the illustrating comprises drawing the branch in a first style when only the first abnormal state is present, drawing the branch in a second style when only the second abnormal state is present, drawing the branch in both the first style and the second style when both the first and second abnormal states are present, and drawing the branch in a third style when neither of the states is present.

3. The method of claim 2, wherein the first style is a first color, the second style is a first line thickness, wherein the third style is a second color and a second line thickness, and wherein the second color is different from the first color and the second line thickness is thinner than the first line thickness.

4. The method of claim 2, wherein the second style is a first color, the first style is a first line thickness, wherein the third style is a second color and a second line thickness, and wherein the second color is different from the first color and the second line thickness is thinner than the first line thickness.

5. The method of claim 2, wherein determining whether the lumen has a first abnormal state and the wall thickness has a second abnormal state comprises:
computing a first ratio between a size of the lumen and a size of the adjacent artery;
computing a second ratio between the wall thickness and the size of the adjacent artery;
determining the lumen to have the first abnormal state when the first ratio is greater than a first bronchial lumen-to-artery (BLA) ratio or less than a second bronchial lumen-to-artery ratio (BLA); and
determining the wall thickness to have the second abnormal state when the second ratio is greater than a first bronchial wall-to-artery ratio (BWA).

6. The method of claim 5, further comprising:
determining the first abnormal state to be a mildly enlarged lumen when the first ratio is greater than the first BLA ratio and less then a third BLA ratio;
determining the first abnormal state to be a moderately enlarged lumen when the first ratio is greater than the third BLA ratio and less than a fourth BLA ratio;
determining the first abnormal state to be a severely enlarged lumen when the first ratio is greater than the fourth BLA ratio; and
determining the first abnormal state to be a narrowed lumen when the first ratio is less than the second predetermined ratio.

7. The method of claim 6, wherein the first style is a first color when the branch had a mildly enlarged lumen, the first style is a second color when the branch has a moderately enlarged lumen, the first style is a third color when the branch has a severely enlarged lumen, the first style is a fourth color when the branch has a narrowed lumen, and the first style is a fifth color when the branch does not have the first abnormal state, wherein the colors differ from one another.

8. The method of claim 5, further comprising:
determining the second abnormal state to be a mildly thickened wall when the second ratio is greater than the first BWA ratio and less then a second BWA ratio;
determining the second abnormal state to be a moderately thickened wall when the second ratio is greater than the second BWA ratio and less then a third BWA ratio; and
determining the second abnormal state to be a severely thickened wall when the second ratio is greater than the third BWA ratio.

9. The method of claim 8, wherein the second style is a first line thickness when the branch has a mildly thickened wall, the second style is a second line thickness when the branch has a moderately thickened wall, the second style is a third line thickness when the branch has a severely thickened wall, and the second style has a fourth line thickness when the branch does not have the second abnormal state, where the first line thickness is greater than the fourth, the second line thickness is greater than the first, and the third line thickness is greater than second.

10. A method of visualizing an airway of a bronchial tree, the method comprising:
generating, by a processor, a tree model from an airway segmentation of a bronchial tree;
determining, by the processor, whether a lumen of a branch of the tree model has a first abnormal state by one of comparing a size of the lumen to an absolute size or comparing a size of the lumen to a size scaled according to a generation of the branch;
determining, by the processor, whether a wall thickness of the branch has a second abnormal state by one of comparing the wall thickness to an absolute thickness or comparing the wall thickness to a thickness scaled according to a generation of the branch; and
illustrating the branch in one of a plurality of visually distinct styles based on the first and second abnormal states.

11. The method of claim 10, wherein the illustrating comprises drawing the branch in a first style when only the first abnormal state is present, drawing the branch in a second style when only the second abnormal state is present, drawing the branch in both the first style and the second style when both the first and second abnormal states are present, and drawing the branch in a third style when neither of the states is present.

12. The method of claim 11, wherein the first style is a first color, the second style is a first line thickness, wherein the third style is a second color and a second line thickness, and wherein the second color is different from the first color and the second line thickness is thinner than the first line thickness.

13. The method of claim 11, wherein the second style is a first color, the first style is a first line thickness, wherein the third style is a second color and a second line thickness, and wherein the second color is different from the first color and the second line thickness is thinner than the first line thickness.

14. A system for displaying an airway in a bronchial tree, the system comprising:
a display;
a memory device for storing a program;
a processor in communication with the memory device, the processor operative with the program to:
generate a tree model from an airway segmentation of a bronchial tree;
determine a lumen, a wall thickness, and an adjacent artery for a branch of the tree model;
determine whether the lumen of the branch has a first abnormal state and the wall thickness of the branch has a second abnormal state based on the adjacent artery; and
illustrate the branch on the display in one of a plurality of visually distinct styles based on the first and second abnormal states.

15. The system of claim 14, further comprising an acquisition device to acquire a 3D image of the bronchial tree from a patient for generating the airway segmentation.

16. The system of claim 15, wherein the acquisition device is a multi-slice computed tomography (MSCT) imaging device or a magnetic resonance (MR) scanner.

17. The system of claim 15, wherein when illustrating the branch, the processor is further operative with the program to draw the branch in a first style when only the first abnormal state is present, draw the branch in a second style when only the second abnormal state is present, draw the branch in both the first style and the second style when both the first and second abnormal states are present, and draw the branch in a third style when neither of the states is present.

18. The system of claim 17, when the first style is a first color, the second style is a first line thickness, the third style is a second color and a second line thickness, and the second color is different from the first color and the second line thickness is thinner than the first line thickness.

19. The system of claim 17, wherein the second style is a first color, the first style is a first line thickness, the third style is a second color and a second line thickness, and the second color is different from the first color and the second line thickness is thinner than the first line thickness.

20. A computer program product for displaying an airway of a bronchial tree, said computer program product comprising:
a computer readable storage medium;
program instructions to:
generate a tree model from an airway segmentation of a bronchial tree;
determine whether a lumen of a branch of the tree model has a first abnormal state by comparing a size of the lumen to an absolute size or comparing a size of the lumen to a size scaled according to a generation of the branch;
determine whether a wall thickness of the branch has a second abnormal state by one of comparing the wall thickness to an absolute thickness or comparing the wall thickness to a thickness scaled according to a generation of the branch; and
illustrate the branch in one of a plurality of visually distinct styles based on the first and second abnormal states,
wherein said program instructions are stored on said computer readable storage medium.
